(19) United States
(12) Patent Application Publication
(10) Pub. No.: US 2014/0257095 A1
(43) Pub. Date: Sep. 11, 2014

(54) SHAPE SENSING INTERVENTIONAL CATHETERS AND METHODS OF USE

(71) Applicant: VOLCANO CORPORATION, San Diego, CA (US)

(72) Inventors: Nathaniel J. Kemp, Concord, MA (US); Timothy K. Glynn, San Marcos, CA (US)

(73) Assignee: VOLCANO CORPORATION, San Diego, CA (US)

(21) Appl. No.: 14/204,033

(22) Filed: Mar. 11, 2014

Related U.S. Application Data
(60) Provisional application No. 61/776,238, filed on Mar. 11, 2013.

(51) Int. Cl. A61B 1/00; A61B 8/12; A61B 5/0205; A61B 5/00; A61B 8/08; A61B 8/00; A61B 5/107; A61B 5/026

(52) U.S. Cl. CPC A61B 1/00167; A61B 5/1076; A61B 8/12; A61B 5/026; A61B 5/0095; A61B 8/483; A61B 1/0017; A61B 8/445; A61B 5/6852; A61B 5/0205
USPC 600/427; 600/587; 600/467; 600/407; 600/483

(57) ABSTRACT

The invention relates to systems and methods for three dimensional imaging of tissue. The invention provides systems and methods to provide a representation of tissue from three-dimensional data that includes intravascular imaging data as well as data representing a shape of the intravascular imaging probe. Device of the invention combine a shape-sensing mechanism with an intravascular intervention catheter.

### FIELD OF THE INVENTION

This application claims the benefit of, and priority to, U.S. Provisional Patent Application Ser. No. 61/776,238, filed Mar. 11, 2013, the contents of which are incorporated by reference.

### FIELD OF THE INVENTION

The invention relates to systems and methods for catheterization with shape-sensing catheters.

### BACKGROUND

Some people are at risk of having a heart attack or stroke due to fatty plaque buildups in their arteries that restrict the flow of blood or even break off and block the flow of blood completely. A number procedures using interventional catheters are hoped to help diagnose and treat these buildups. For example, angioplasty involves inserting a guidewire into a patients' vessels and guiding it to the affected site. A physician tries to guide the wire by twisting and manipulating the proximal end that sits outside the patient. The guidewire is meant to help in a number of treatment options. For example, an imaging catheter (e.g., with an ultrasound or optical imaging sensor) can be used to visualize the affected site. Forward-looking ultrasound can be used to measure blood velocity by Doppler. If the affected blood vessel is severely narrowed by plaque, a catheter can be used in treatment procedures. In angioplasty procedures, a balloon or stent is delivered to the affected site in hopes of opening up the narrowed vessel. If the affected site is blocked, a tool can be used to cut through the blockage.

Unfortunately, these diagnostic and treatment procedures are imperfect. A patient's vasculature is defined by a super-fine network of very small veins and arteries that branch extensively. Existing methods for knowing the position and shape of the catheter include radiopaque markers and other imprecise x-ray based techniques. Since treating a plaque buildup requires positioning the catheter precisely and avoiding damage to the walls of the vessels, the lack of precise knowledge of a shape of a catheter renders much of the vasculature off-limits to existing procedures. Additionally, intravascular images and measurements can be distorted in counter-intuitive ways by catheter orientation. For example, where an intravascular imaging procedure shows a blood vessel wall on a computer monitor, a human viewer tends to interpret the image as though the imagine catheter and blood vessel are parallel and co-axial. Without information about the catheter position, the observer does not have enough information to perform the linear transformations to correct for distortions in the image.

### SUMMARY

The invention provides systems and methods that combine an interventional catheter with a shape-sensing mechanism so that the catheter operates to determine its own shape while studying or treating tissue. Since the catheter includes a mechanism that gives information about a present shape of the catheter, the interventional procedure can be guided by that shape information. For example, catheter can provide intravascular imaging, blood pressure or flow measurements, or tools for crossing a chronic total occlusion while shape-sensing elements within the catheter inform the imaging, measurement, or treatment procedure. Since a surgeon can perform an interventional catheterization procedure with precise information about the present shape of the catheter, the catheter can be guided with great precision to the target of the procedure while inadvertent contact with other parts of the patient's tissue is avoided. Additionally, images or measurements can be provided along with information about the present shape or position of the catheter. A computer device that receives and processes those data can transform the image to correct for distortions associated with catheter shape and positioning. The system can the display (e.g., on a computer monitor) an intravascular image that represents the actual shape and disposition of the patient's tissues. Thus, a shape-sensing catheter of the invention provides much greater precision and fidelity when performing intravascular interventions, allowing surgeons to access a great extent of circulatory system and view faithful and accurate representations of the patient's blood vessels.

In certain aspects, the invention provides a method for examining tissue that includes using an intravascular probe to evaluate bodily material and determining a shape of the probe using a shape-sensing mechanism of the probe. Preferably, evaluating the bodily material comprises obtaining and storing in a tangible memory coupled to a processor within a computing device a three-dimensional data set representing tissue. In some embodiments, the probe is part of an OCT or ultrasound image collection system and the three-dimensional data set comprises B-scans comprising A-lines. The shape-sensing mechanism may include one or more fiber cores and an array of fiber Bragg gratings disposed within each fiber core (e.g., the array of fiber Bragg gratings are substantially collocated along each fiber core). Further, the array may include at least one hundred fiber Bragg gratings, the shape-sensing mechanism may include three non-coplanar optical fibers, or both. Evaluating the bodily material can include measuring fractional flow reserve, performing an intra-vascular ultrasound imaging operation, photoacoustic imaging, or a combination thereof. In some embodiments, evaluating the bodily material comprises performing an intravascular imaging operation to obtain a three-dimensional data set representing tissue and using the determined shape to present a provide a three-dimensional view of the three-dimensional data set representing tissue.

In some embodiments, the probe comprises an imaging catheter and the method further includes performing, using the catheter, an intravascular imaging operation to obtain a three-dimensional data set representing tissue and using the determined shape to correct a distortion in the three-dimensional data set.

The intravascular probe may include an optical fiber and the shape-sensing mechanism may include the optical fiber (e.g., with one or more fiber Bragg gratings therein). The method may further include imaging tissue within a vessel using the optical fiber.

In some aspects, the invention provides a catheter-based sensing apparatus that has an elongated catheter body, a fiber optic member extending along the body and configured to detect a shape of the body, and an intravascular sensing device. An optical connection to an imaging engine with a memory coupled to a processor and operable to receive shape information from the fiber optic member and an intravascular image of tissue from the sensing device may be included. The apparatus may use a display unit operably coupled to the imaging engine and operable to display a 3- or 4-dimensional image of tissue. A 4-dimensional image of tissue may be displayed by showing a depiction of three dimensions of the image of tissue on a screen with coordinate axes, rotating the depiction according to user input, and depicting a fourth dimension of the image by changing the depiction on the screen as time elapses.

Aspects of the invention provide a system for examining tissue that includes an intravascular probe configured to evaluate bodily material and a shape-sensing mechanism configured to determine a shape of the probe using the probe. Preferably, the probe includes an imaging mechanism and the system further includes a tangible memory coupled to a processor within a computing device operable to receive and store a three-dimensional data set representing tissue captured by the imaging mechanism. In some embodiments, the probe is part of an OCT or ultrasound image collection system (e.g., operable to capture a three-dimensional data set comprises B-scans made up of A-lines). The shape-sensing mechanism may include at least two fiber cores and an array of fiber Bragg gratings disposed within each fiber core (e.g., substantially collocated along each fiber core).

Efforts have been made to develop shape-sensing optical systems. For example, U.S. Pat. No. 6,256,090 to Chen and U.S. Pat. No. 7,781,724 to Childers, both incorporated by reference, both may be modified to provide at least a portion of the shape-sensing mechanisms and method of an intravascular catheterization device or method of the invention. Systems and methods of the invention provide three-dimensional image data sets of a patient's tissue using an intravascular catheter that also includes a shape-sensing mechanism, such as an array of fiber Bragg gratings for strain sensing.

In certain embodiments, a three-dimensional image data set includes a set of A scan lines as captured by a medical imaging system, such as an OCT system. A set of A scan lines may be grouped into B-scans, which can be used to compose a tomographic view of tissue. Systems and methods of the invention operate in OCT or ultrasound imaging systems. A user can select data from within a three-dimensional data set by interacting with a graphical user interface (GUI), for example, by operating a computer pointing mechanism such as a mouse or touch-screen. A montage (e.g., a representation including the image, the longitudinal image, and the indicator of the relationship between the image and the longitudinal image) can be presented to a user by any means such as rendering a montage as a display (e.g., within a GUI) or saving it in a file in a storage medium. Methods of the invention further include displaying a 3D or 4D image to a user. In some embodiments, an image is displayed in sequence, among a plurality of images, to create an animation simulating motion through the tissue, such as traveling down a lumen, thereby showing a 3D display. Information about the instantaneous shape of the catheter gives accuracy and precision to the displayed image and also corrects for distortion. A user may select an image by choosing a point within the animation, for example, by pressing a key (e.g., space bar) while an animation is playing.

In certain aspects, the invention provides a device for creating an image of tissue comprising a memory coupled to a processor and configured to obtain a three-dimensional data set representing tissue, receive data indicating a shape of an imaging device at the moment it captured the three-dimensional data set, and automatically provide, using the processor, a representation comprising the image that accurately represents a 3D (or 4D) shape of the tissue. The device can repeat these steps, for instance, automatically or responsive to user input.

A device of the invention can be a computer, for example, with a monitor, keyboard, and mouse or trackpad, through which a user interacts with imaging system data. Exemplary devices of the invention include an input mechanism configured to be operably coupled to receive input from an OCT or ultrasound imaging device. A monitor can display an image from the data set or a video. A computing device generally includes a tangible, non-transitory storage medium.

### BRIEF DESCRIPTION OF THE DRAWINGS

FIG. 1 shows a segment of a vessel having a feature of interest.

FIG. 2 shows a cross-section of the vessel through the feature of interest.

FIG. 3 illustrates a differential beam path OCT system.

FIG. 4 shows the components for shape sensing.

FIG. 5 diagrams an imaging engine according to certain embodiments.

FIG. 6 shows light path in a differential beam path system.

FIG. 7 shows components of a patient interface module.

FIG. 8 shows a path that image collection follows during OCT.

FIG. 9 depicts an array of A scan lines.

FIG. 10 shows the positioning of A scans within a vessel.

FIG. 11 illustrates a set of A scans.

FIG. 12 shows the set of A scans.

FIG. 13 shows a longitudinal plane through a vessel with A-scan lines.

FIG. 14 shows a longitudinal plane through a vessel.

FIG. 15 illustrates obtaining an image longitudinal display.

FIG. 16 shows a portion of a sample vessel.

FIG. 17 is a cross-sectional view of the sample vessel.

FIG. 18 shows a display including an image of the vessel provided by the invention.

FIG. 19 depicts a fiber optic position and shape sensing device.

FIG. 20 depicts components of systems of certain embodiments.

FIG. 21 illustrates the local coordinate system.

FIG. 22 shows a coordinate system.

FIG. 23 defines a geometry of a bend and a bend radius.

FIG. 24 illustrates curvature of an object.

FIG. 25 shows geometrically differing lengths of core segments.

FIG. 26 is a 2D projection of a vessel border.

FIG. 27 is a 3D representation of a vessel border.

FIG. 28 diagrams a system of the invention.

### DETAILED DESCRIPTION

The invention generally relates to systems and methods for examining tissue. The invention allows a user to obtain a three dimensional image of tissue in the form of a three dimensional data set representing tissue and simultaneously collect information about the shape of the imaging probe. In some embodiments, the invention provides a computing device operable to obtain a three-dimensional data set representing tissue, receive data about the shape of the imaging device that captured the three-dimensional data set, and provide a representation comprising the three-dimensional data set transformed according to the shape of the imaging device.

The invention provides interventional catheters with shape sensing capabilities. In some embodiments, the interventional catheter is an optical coherence tomography (OCT) intravascular imaging catheter with 3D Shape Sensing Capability. The invention provides a catheter or probe-based OCT imaging apparatus (or other catheter based sensing/imaging device) with capability of detecting conformational shape of the probe in 4 dimensions (3 spatial dimensions plus time). Different architectures are disclosed which allow sharing system hardware components between OCT and shape-sensing instrument to reduce size and cost. The use of 3D probe shape information allows the system to analyze and display captured 3D OCT image volumes in a true 3D spatial orientation (rather than a 2D linear projection as is typical). The 3D shape-sensing approach may be based on distributed Fiber Bragg Grating strain sensors and interferometric interrogation.

The interferometric interrogation technique (Optical Frequency Domain Reflectometry, OFDR) used to perform 3D shape-sensing is similar to the technique used in swept-source OCT (aka Optical Frequency Domain Imaging, OFDI), and thus potential exists for combining these two technologies into a single combined system and sharing some of the system hardware components.

In certain embodiments, systems and methods of the invention combine the OCT instrument and shape-sensing instrument into a single system and leverage components which can be shared between the two instruments in order to save cost, space, or other valuable resource. Systems and methods of the invention also include the combination of any other catheter based sensing (e.g. FFR) or imaging device (e.g. IVUS) with the shape sensing technology and leveraging common components. These other technologies may have fewer shared components given that they do not employ a light source or a fiber optic catheter to collect data. Additionally, they may be able to share other electronic and hardware components such as DAQ boards, FPGAs, etc.

The invention provides methods for using 3D shape sensing technology to detect the shape of an imaging catheter during image acquisition, and then analyzing and/or displaying/rendering the acquired images in a 3D configuration based on the sensed shape data (rather than a 2D linear projection). This method is applicable whether the imaging modality and shape-sensing instruments are combined or separate, and regardless of the technology used for shape-sensing.

Using the shape sensing data, distortions due to catheter eccentricity or the angle of the catheter inside the lumen may be corrected. In addition to improving the 3D display, the shape sensing data may also be used to adjust the tomographic and ILD displays. Examples of how this data may be applied to correct for distortions is described in U.S. Pat. No. 7,024,025 to Sathyanarayana; U.S. Pat. No. 5,872,829 to Wischmann; U.S. Pub. 2012/0262720 to Brown; U.S. Pub. 2012/0224751 to Kemp; U.S. Pub. 2008/0085041 to Breeuwer, the contents of each of which are incorporated by reference. Combining the OCT, IVUS, Photoacoustic, FFR, or other instrument and shape-sensing instrument into a single system leverages components that can be shared between the two instruments in order to save cost, space, or other valuable resources. Using shape sensing technology to register and/or display OCT images in 3D provides much more informative displays and data sets than prior art methods systems.

Systems and methods of the invention have application in intravascular imaging methodologies such as intravascular ultrasound (IVUS) and optical coherence tomography (OCT) among others that produce a three-dimensional image of a vessel.

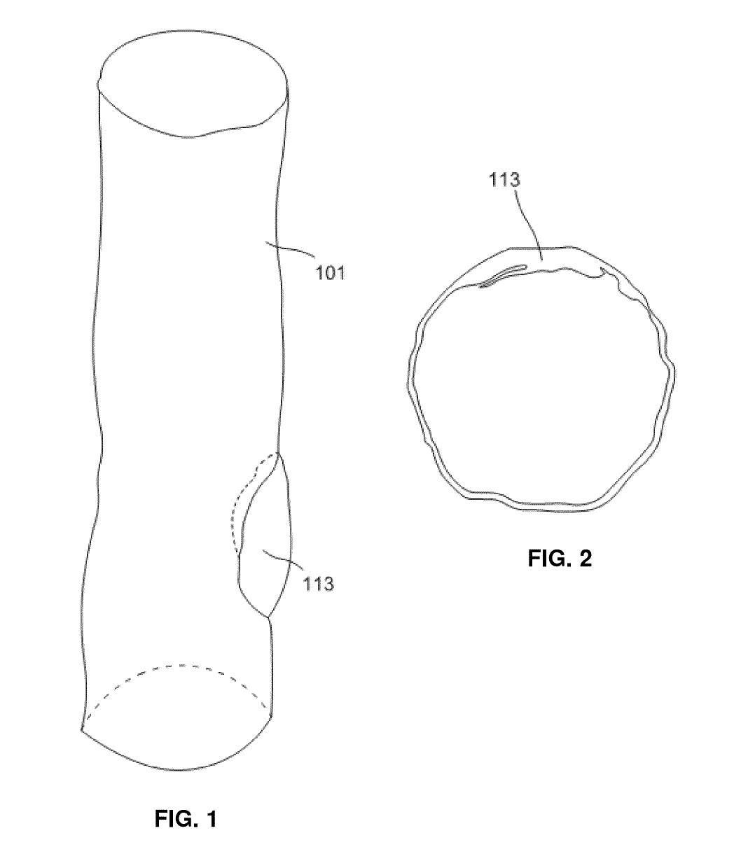

FIG. 1 shows a segment of a vessel 101 having a feature 113 of interest.

FIG. 2 shows a cross-section of vessel 101 through feature 113. In certain embodiments, intravascular imaging involves positioning an imaging device near feature 113 and collecting data representing a three-dimensional image.

Any three-dimensional imaging system may be used in systems and methods of the invention including, for example, IVUS; magnetic resonance imaging; elastographic techniques such as magnetic resonance elastography or transient elastography systems such as FibroScan by Echosens (Paris, France); electrical impedance tomography; and OCT. In certain embodiments, systems and methods of the invention include processing hardware configured to interact with more than one different three dimensional imaging system so that the tissue imaging devices and methods described here in can be alternatively used with OCT, IVUS, or other hardware.

Various lumen of biological structures may be imaged with aforementioned imaging technologies in addition to blood vessels, including, but not limited to, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, vagina, uterus and fallopian tubes, structures of the urinary tract including urinary collecting ducts, renal tubules, ureter, and bladder, and structures of the head and neck and pulmonary system including sinuses, parotid, trachea, bronchi, and lungs.

In an exemplary embodiment, the invention provides a system for capturing a three dimensional image by OCT. Commercially available OCT systems are employed in diverse applications such as art conservation and diagnostic medicine, e.g., ophthalmology. OCT is also used in interventional cardiology, for example, to diagnose coronary artery disease. OCT systems and methods are described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety.

In OCT, a light source delivers a beam of light to an imaging device to image target tissue. Within the light source is an optical amplifier and a tunable filter that allows a user to select a wavelength of light to be amplified. Wavelengths commonly used in medical applications include near-infrared light, for example between about 800 nm and about 1700 nm.

Generally, there are two types of OCT systems, common beam path systems and differential beam path systems, that differ from each other based upon the optical layout of the systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in its entirety.

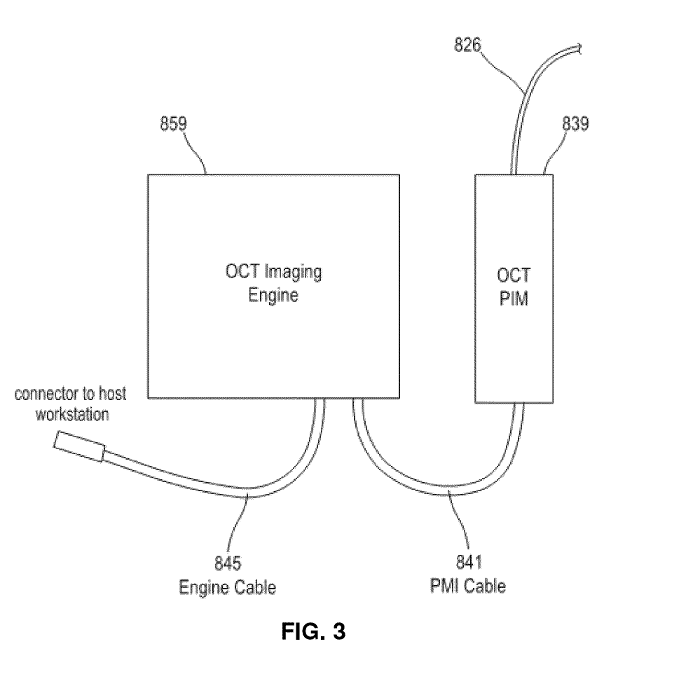

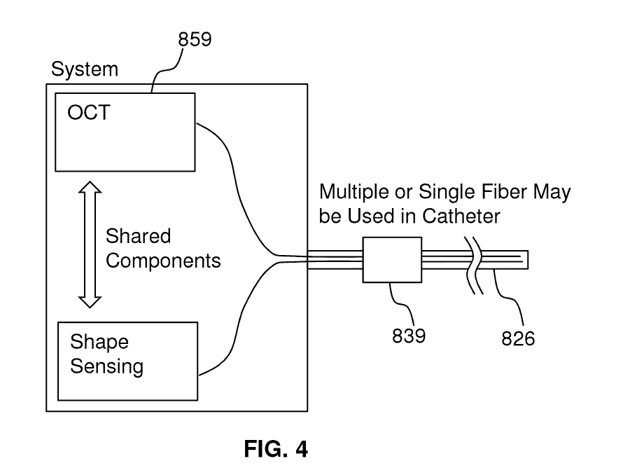

FIG. 3 illustrates a differential beam path OCT system with intravascular imaging capability and shape-sensing capability as provided by the invention. For intravascular imaging, a light beam is delivered to the vessel lumen via a fiber-optic based imaging catheter 826. The imaging catheter is connected through hardware to software on a host workstation. The hardware includes an imagining engine 859 and a handheld patient interface module (PIM) 839 that includes user controls. The proximal end of the imaging catheter is connected to PIM 839, which is connected to an imaging engine as shown in FIG. 3A.

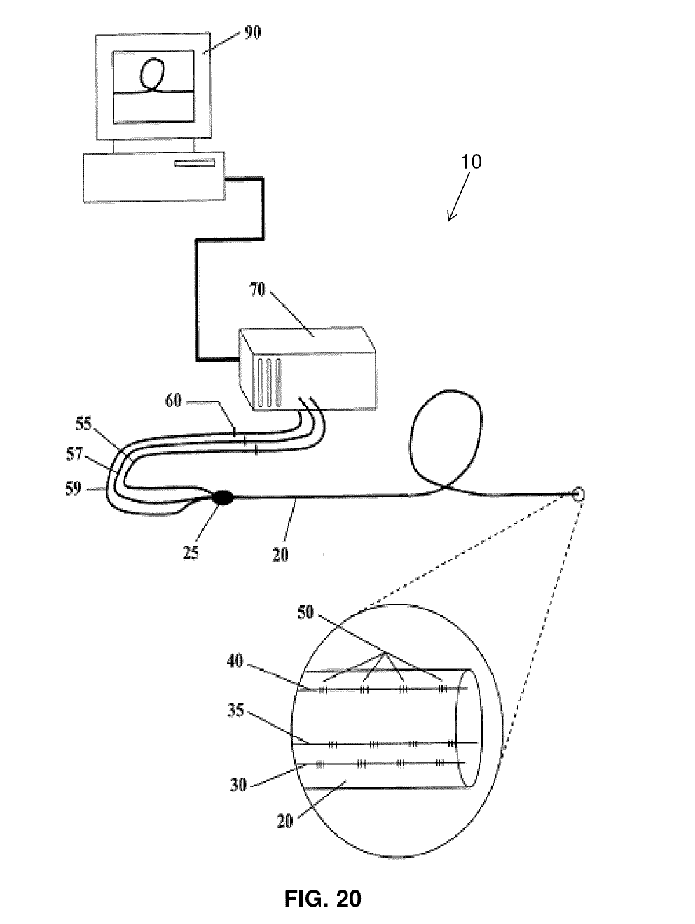

FIG. 4 shows the components for shape sensing included in the system. Shape sensing components are discussed in greater detail below with reference to FIGS. 19-27.

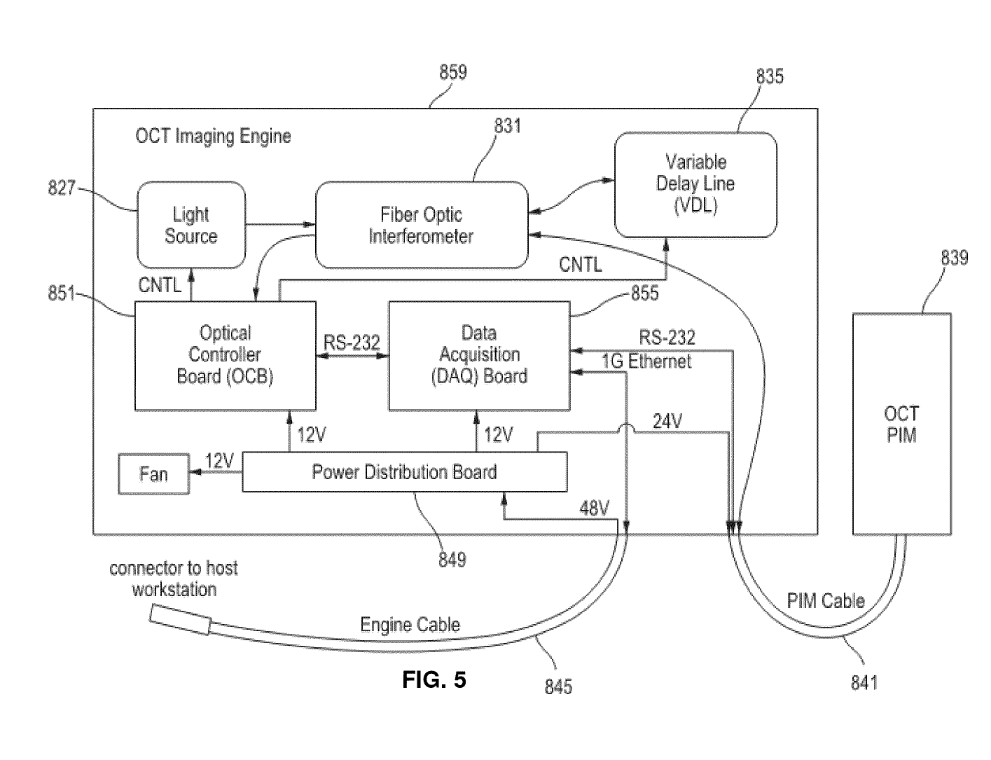

FIG. 5 shows imaging engine 859 (e.g., a bedside unit), which houses a power supply 849, light source 827, interferometer 831, and variable delay line 835 as well as a data acquisition (DAQ) board 855 and optical controller board (OCB) 851 as well as one or more processor for controlling and analyzing the shape sensing mechanism. A PIM cable 841 connects the imagine engine 859 to the PIM 839 and an engine cable 845 connects the imaging engine 859 to the host workstation. It is noted that DAQ 855, OCB 851, PIM cable 841, OCT PIM 839, power board 849, light source 827, or any combination thereof can be used for shape-sensing (discussed below) as well as OCT (discussed immediately below).

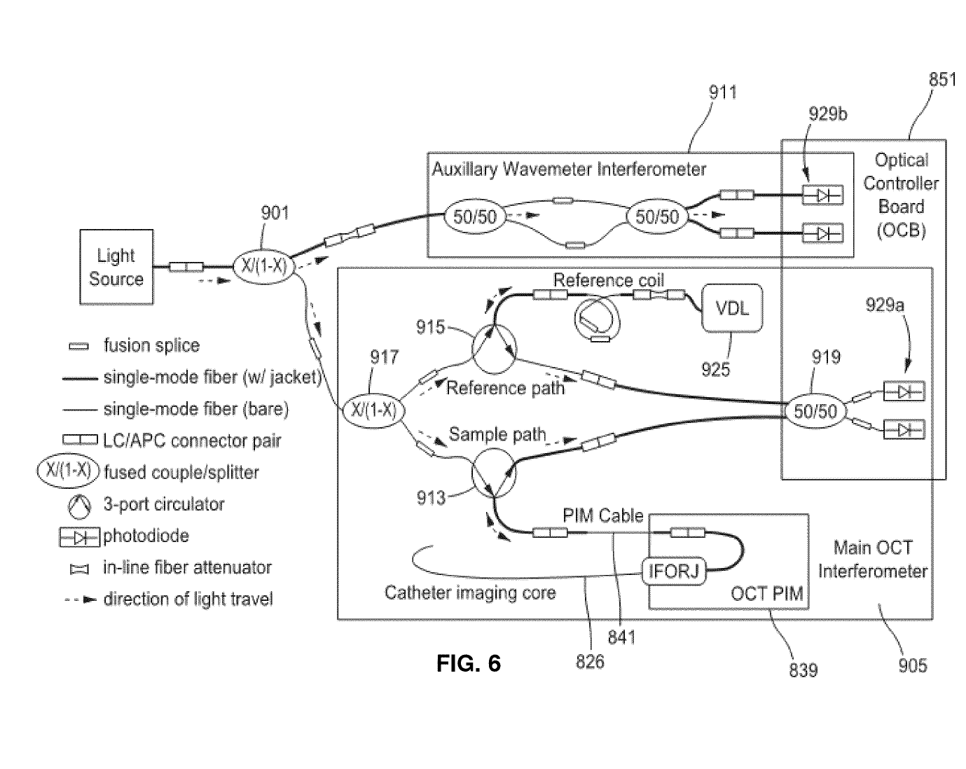

FIG. 6 shows light path in a differential beam path system according to an exemplary embodiment of the invention. Light for image capture originates within the light source 827. This light is split between an OCT interferometer 905 and an auxiliary, or “clock”, interferometer 911. Light directed to the OCT interferometer is further split by splitter 917 and recombined by splitter 919 with an asymmetric split ratio. The majority of the light is guided into the sample path 913 and the remainder into a reference path 915. The sample path includes optical fibers running through the PIM 839 and the imaging catheter 826 and terminating at the distal end of the imaging catheter where the image is captured. Additionally, imaging catheter 826 includes a mechanism for shape sensing. While discussed in greater detail below, a shape-sensing mechanism may include one or more optical fibers (i.e., fiber cores), any of which may include a plurality of fiber Bragg gratings. Light sent to OCB 851 can include light from the shape sensing mechanism that reveals strain within imaging catheter 826 and thus can be digitized and used to determine a shape of catheter 826 at the moment the light traveled therethrough.

Typical intravascular OCT involves introducing the imaging catheter into a patient's target vessel using standard interventional techniques and tools such as a guide wire, guide catheter, and angiography system.

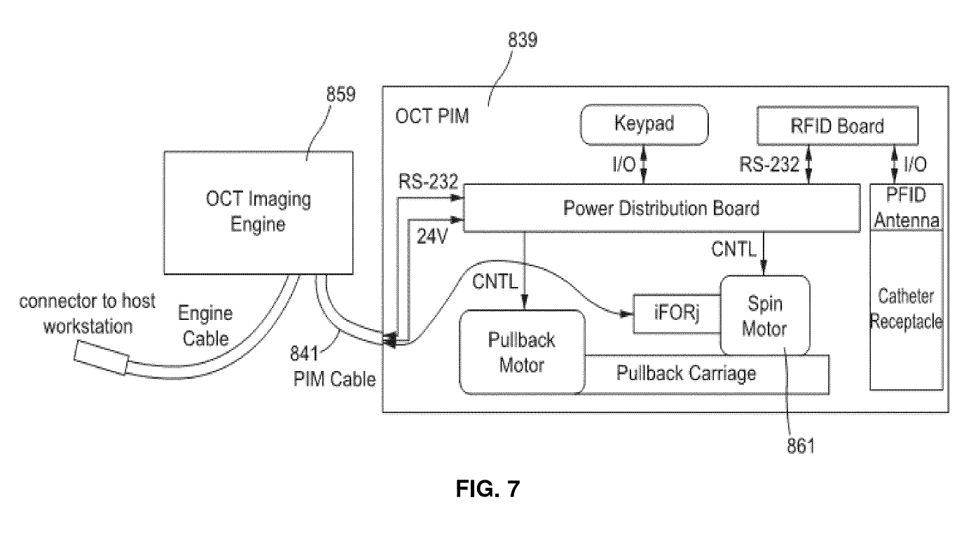

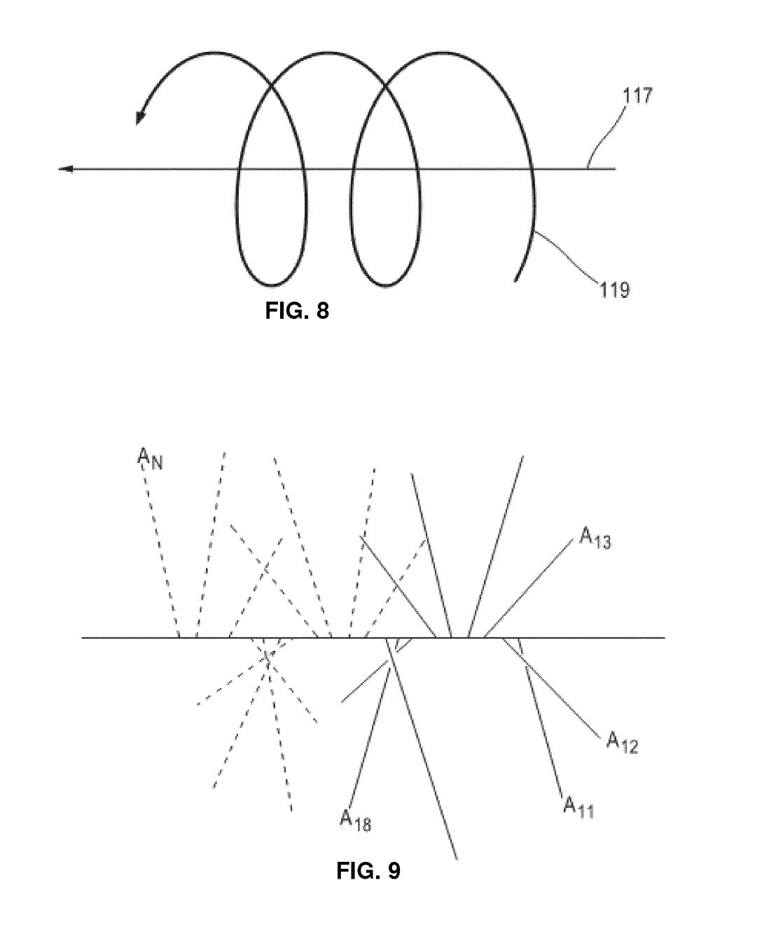

FIG. 7 shows spin motor 861, which drives rotation while translation is driven by pullback motor 865. This results in a motion for image capture described by FIG. 8.

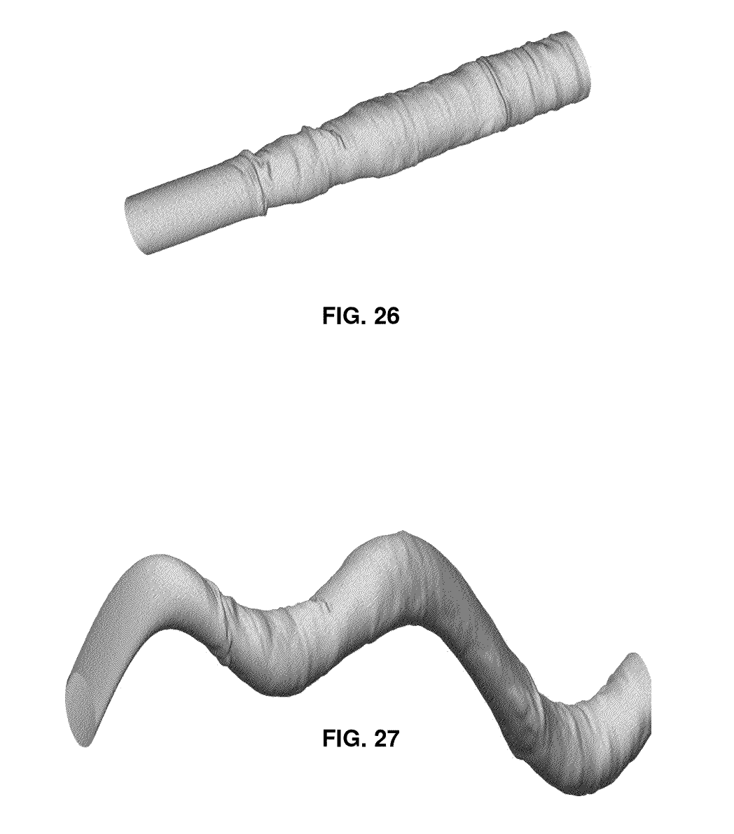

FIG. 8 shows a path 119 that image collection follows during OCT. Path 119 is substantially helical about axis 117. However, FIG. 8 depicts axis 117 as linear and one important insight of the invention is that an intravascular catheter axis may generally be not substantially linear but may in fact be contoured according to a shape of a patient's vessels. In fact, since the catheter according to the invention collects precise shape of itself, it is all the more appropriate to show an axis in the simplifying embodiment as linear, since the actual shape is captured and stored (e.g., in a tangible, non-transitory memory), allowing the axis to be transformed according to the shape information to present a realistic 3 dimensional depiction of the patient's tissue (e.g., an image like FIG. 26 can be transformed into an image like FIG. 27). Accordingly, FIGS. 8-15 may appear to depict the axis of the image catheter as straight, but that is for ease of illustration only. Systems and methods of the invention actually capture the 3D shape of the axis.

During OCT imaging, blood in the vessel is temporarily flushed with a clear solution for imaging. When operation is triggered from the PIM or control console, the imaging core of the catheter rotates while collecting image data.

FIG. 9 depicts an array of A scan lines that the inner core sends light into the tissue. The inner core detects reflected light.

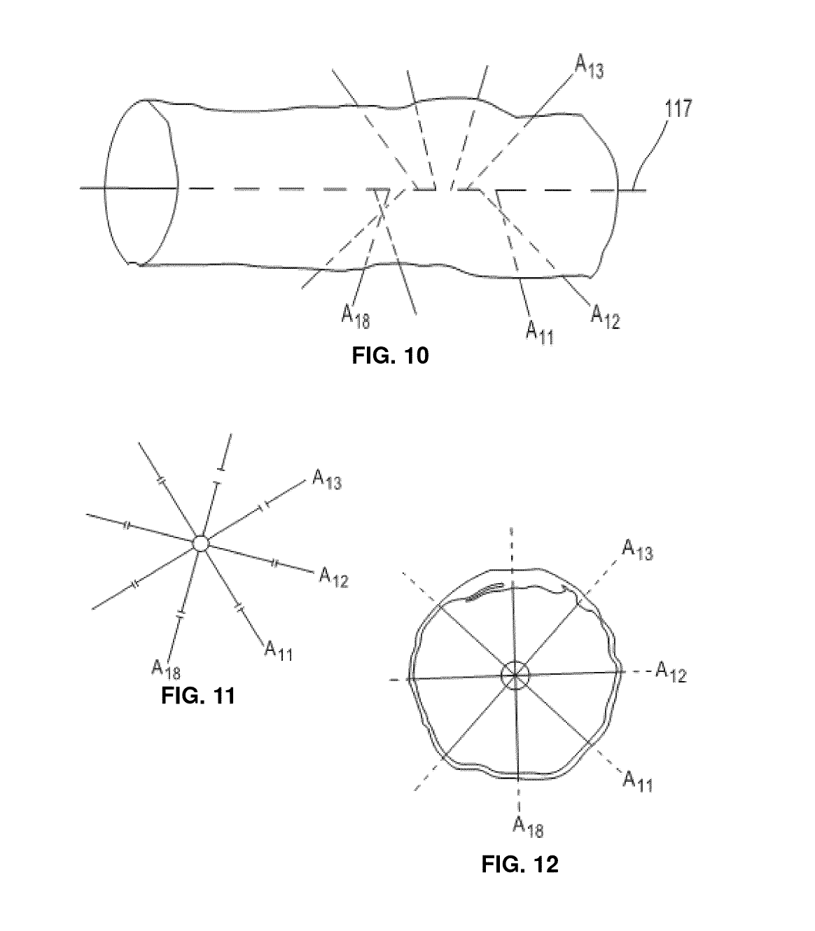

FIG. 10 shows the positioning of A scans within a vessel. Each place where one of A scans A11, A12, . . . , AN intersects a surface of a feature within vessel 101 (e.g., a vessel wall) coherent light is reflected and detected. Catheter 826 translates along axis 117 being pushed or pulled by pullback motor 865.

The reflected, detected light is transmitted along sample path 913 to be recombined with the light from reference path 915 at splitter 919 (FIG. 6). A variable delay line (VDL) 925 on the reference path uses an adjustable fiber coil to match the length of reference path 915 to the length of sample path 913. The reference path length is adjusted by a stepper motor translating a minor on a translation stage under the control of firmware or software. The free-space optical beam on the inside of the VDL 925 experiences more delay as the minor moves away from the fixed input/output fiber.

The combined light from splitter 919 is split into orthogonal polarization states, resulting in RF-band polarization-diverse temporal interference fringe signals. The interference fringe signals are converted to photocurrents using PIN photodiodes 929a, 929b, . . . on the OCB 851 as shown in FIG. 6. The interfering, polarization splitting, and detection steps are done by a polarization diversity module (PDM) on the OCB. Signal from the OCB is sent to the DAQ 855, shown in FIG. 5. The DAQ includes a digital signal processing (DSP) microprocessor and a field programmable gate array (FPGA) to digitize signals and communicate with the host workstation and the PIM. The FPGA converts raw optical interference signals into meaningful OCT images. The DAQ also compresses data as necessary to reduce image transfer bandwidth to 1 Gbps (e.g., compressing frames with a lossy compression JPEG encoder).

Data is collected from A scans A11, A12, . . . , AN and stored in a tangible, non-transitory memory. A set of A scans generally corresponding to one rotation of catheter 826 around axis 117 collectively define a B scan.

FIG. 11 illustrates a set of A scans A11, A12, . . ., A18 used to compose a B scan according to certain embodiments of the invention. These A scan lines are shown as would be seen looking down axis 117 (i.e., longitudinal distance between then is not shown). In certain embodiments, the data collected from the A scans provide a three-dimensional data set representing tissue. In some embodiments, a device of the invention includes an OCT imaging system and obtains a three-dimensional data set through the operation of OCT imaging hardware. In some embodiments, a device of the invention is a computing device such as a laptop, desktop, or tablet computer, and obtains a three-dimensional data set by retrieving it from a tangible storage medium, such as a disk drive on a server using a network or as an email attachment.

While eight A scan lines are illustrated in FIG. 11, typical OCT applications can include between 300 and 1,000 A scan lines to create a B scan (e.g., about 660). Reflections detected along each A scan line are associated with features within the imaged tissue. Reflected light from each A scan is combined with corresponding light that was split and sent through reference path 915 and VDL 925 and interference between these two light paths as they are recombined indicates features in the tissue.

The data of all the A scan lines together represent a three-dimensional image of the tissue. The data of the A scan lines can be used to create an image of a cross section of the tissue, sometimes referred to as a tomographic view.

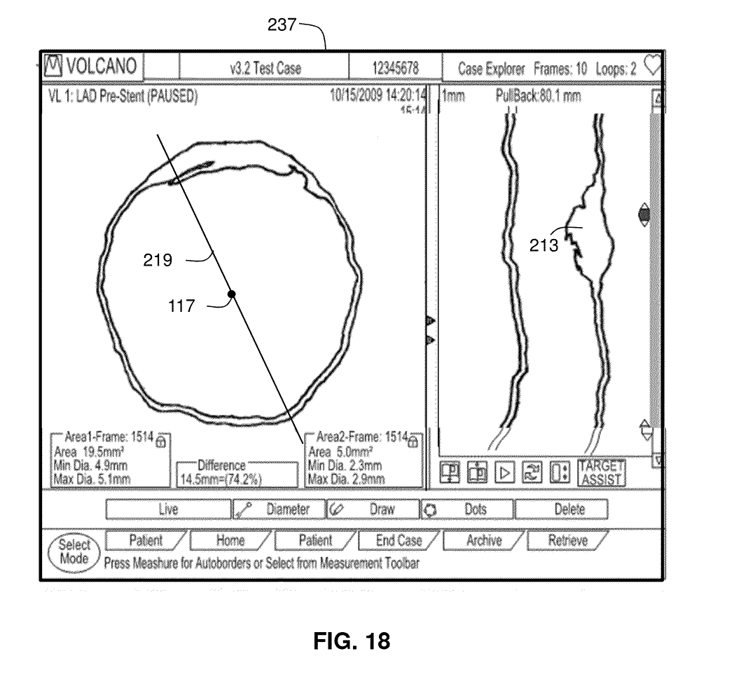

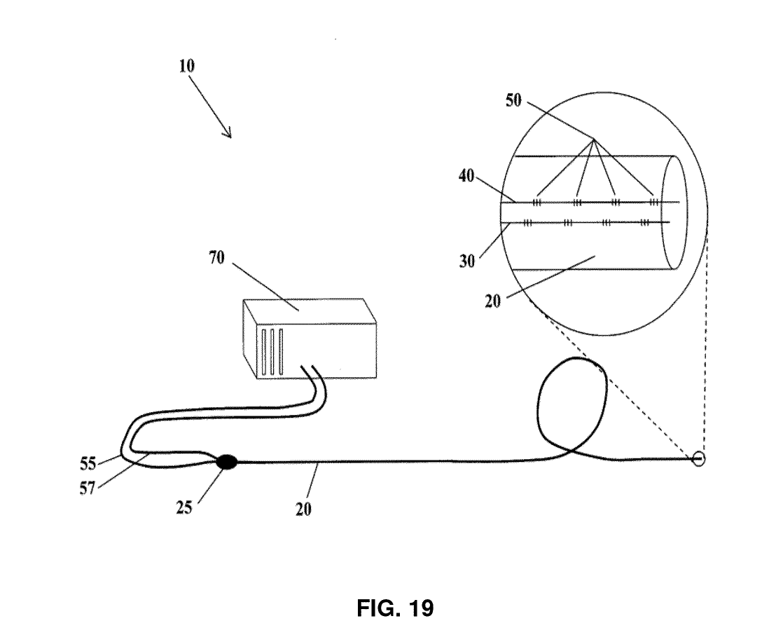

FIG. 12 shows the set of A scans shown in FIG. 11 within a cross section of a vessel. A B scan can be represented as a visual depiction of a cross section of a vessel (see left side of FIG. 18).

Where a tomographic view generally represents an image as a planar view across a vessel or other tissue (i.e., substantially normal to axis 117), an image can also be represented as a planar view along a vessel (i.e., axis 117 lies substantially within the plane of the view).

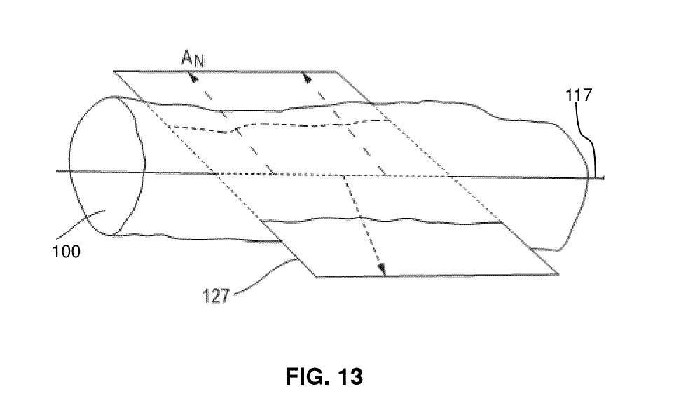

FIG. 13 shows a longitudinal plane 127 through a vessel 101 including several A scans. Such a planar image along a vessel is sometimes referred to as an in-line digital view or image longitudinal display (ILD). As shown in FIG. 13, plane 127 generally comprises data associated with a subset of the A scans.

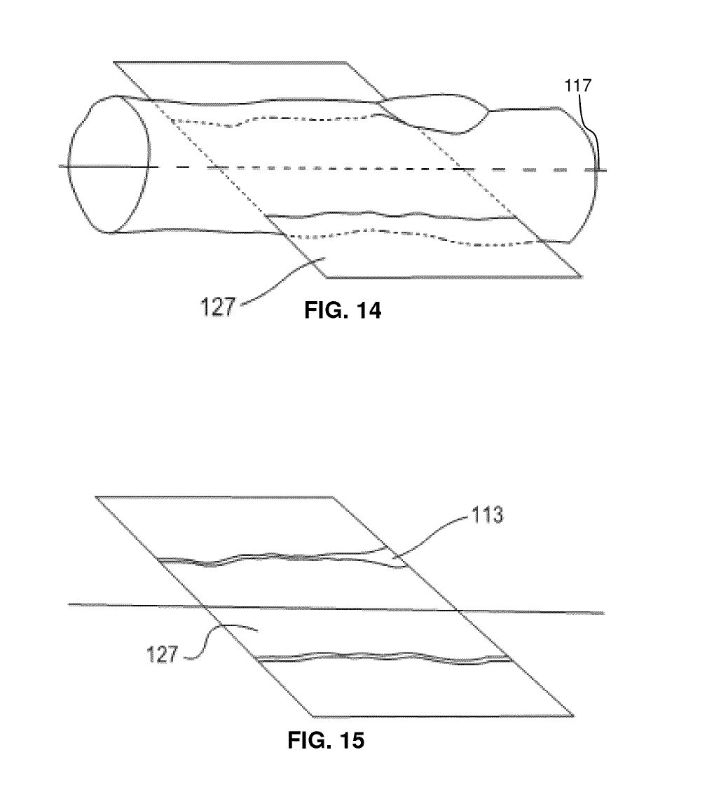

FIG. 14 shows a longitudinal plane through a vessel drawn without the A scan lines (of FIG. 13) to assist in visualizing plane 127 comprising axis 117. As used herein, a longitudinal image preferably is an image of tissue that is substantially orthogonal to a cross-sectional view. Where an image capture system operates via a one-dimensional motion of an image capture device, a longitudinal image lies in a plane that is substantially parallel to a vector defined by the one-dimensional motion of the image capture device. An ILD is a longitudinal image that includes the axis of translation of the image capture device. For example, in FIG. 14, plane 127 corresponds to an ILD due to the fact that plane 127 includes axis 117. A longitudinal image is an image in a plane substantially parallel to plane 127.

The data of the A scan lines is processed according to systems and methods of the inventions to generate images of the tissue. By processing the data appropriately (e.g., by fast Fourier transformation), a two-dimensional image can be prepared from the three dimensional data set. Systems and methods of the invention provide one or more of a tomographic view, ILD, or both.

FIG. 15 is a perspective view of an idealized plane shown including an exemplary ILD in the same perspective as the longitudinal plane shown in FIGS. 13 and 14. The ILD shown in FIG. 15 can be presented by systems and methods described herein, for example, as shown in the right area of the display illustrated in FIG. 18.

The image shown in FIG. 15 showing feature 113 and the image shown in FIG. 12 represent planes through tissue 101 that have a spatial relationship to each other. To the extent that the planes have a spatial relationship, the images in FIGS. 12 and 15 can be described as having a spatial relationship. Here, the image in FIG. 12 is substantially orthogonal to the image in FIG. 15. In general herein, in three-dimensional imaging technologies, a tomographic view from a data set is substantially orthogonal to an ILD from the same data set, unless otherwise specified.

Systems and methods of the invention are operable with any compatible method of generating a three-dimensional image of tissue. In certain embodiments, the invention provides systems and methods for providing a montage of images from a three-dimensional data set generated using intravascular ultrasound (IVUS). IVUS uses a catheter with an ultrasound probe attached at the distal end. The proximal end of the catheter is attached to computerized ultrasound equipment. To visualize a vessel via IVUS, angiographic techniques are used and the physician positions the tip of a guide wire, usually 0.36 mm (0.014″) diameter and about 200 cm long. The physician steers the guide wire from outside the body, through angiography catheters and into the blood vessel branch to be imaged.

The ultrasound catheter tip is slid in over the guide wire and positioned, again, using angiography techniques, so that the tip is at the farthest away position to be imaged. Sound waves are emitted from the catheter tip (e.g., in about a 20-40 MHz range) and the catheter also receives and conducts the return echo information out to the external computerized ultrasound equipment, which constructs and displays a real time ultrasound image of a thin section of the blood vessel currently surrounding the catheter tip, usually displayed at 30 frames/second image.

The guide wire is kept stationary and the ultrasound catheter tip is slid backwards, usually under motorized control at a pullback speed of 0.5 mm/s. Systems for IVUS are discussed in U.S. Pat. No. 5,771,895; U.S. Pub. 2009/0284332; U.S. Pub. 2009/0195514 A1; U.S. Pub. 2007/0232933; and U.S. Pub. 2005/0249391, the contents of each of which are hereby incorporated by reference in their entirety.

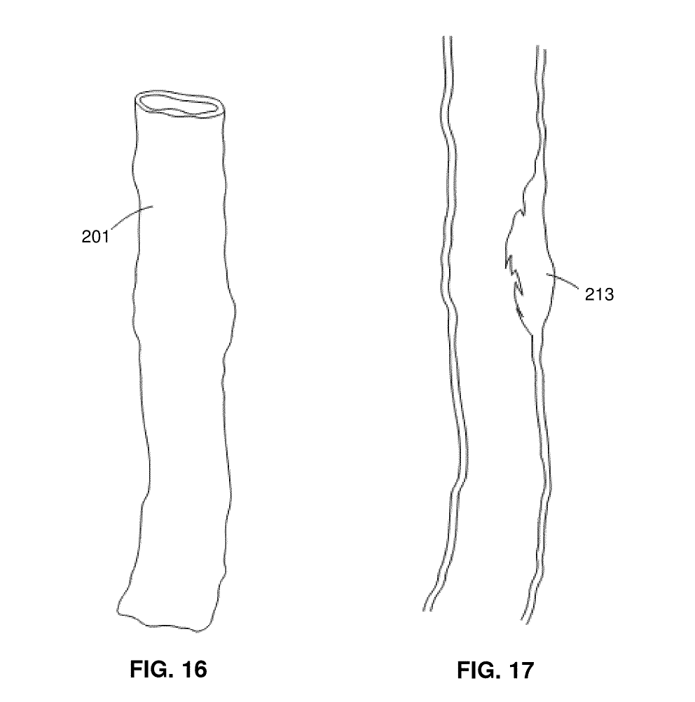

FIG. 16 shows a portion of a vessel 201. Systems and methods of the invention provide an operator with images of tissue such as, for example, the portion of vessel 201 that is shown in FIG. 16.

FIG. 17 is a cross-sectional view of the vessel shown in FIG. 17, presented for reference in subsequent discussion. As can be seen in FIGS. 17 and 18, example target tissue 201 includes a region of interest 213. An operator may or may not have a priori knowledge of the existence of region 213.

In certain embodiments, a system for three dimensional imaging is operated to capture an image of tissue 201. An electronic apparatus within the system (e.g., PC, dedicated hardware, or firmware) stores the three dimensional image in a tangible, non-transitory memory and renders a display (e.g., on a screen or computer monitor) including at least a first image of tissue 201.

FIG. 18 is an illustration of a display 237 including an image of the vessel shown in FIGS. 16-17, as rendered by a system of the invention. The images included in display 237 in FIG. 18 are rendered in a simplified style for ease of understanding. A system of the invention may render a display as shown in FIG. 18, or in any style known in the art (e.g., with or without color).

In certain embodiments, display 237 is rendered within a windows-based operating system environment, such as Windows, Mac OS, or Linux or within a display or GUI of a specialized system. Display 237 can include any standard controls associated with a display (e.g., within a windowing environment) including minimize and close buttons, scroll bars, menus, and window resizing controls. Elements of display 237 can be provided by an operating system, windows environment, application programing interface (API), web browser, program, or combination thereof (for example, in some embodiments a computer includes an operating system in which an independent program such as a web browser runs and the independent program supplies one or more of an API to render elements of a GUI). Display 237 can further include any controls or information related to viewing images (e.g., zoom, color controls, brightness/contrast) or handling files comprising three-dimensional image data (e.g., open, save, close, select, cut, delete, etc.). Further, display 237 can include controls (e.g., buttons, sliders, tabs, switches) related to manipulating images within display 237 (e.g., rotate, select, invert selection, save selection, preview montage, save montage (JPG, TIF, etc.), export montage (PPT, XCF, PSD, SVG, etc.), etc.).

In certain embodiments, display 237 includes controls related to three dimensional imaging systems that are operable with different imaging modalities. For example, display 237 generally may include start, stop, zoom, save, etc., buttons, and be rendered by a computer program that interoperates with OCT or IVUS modalities. Thus display 237 can display an image to a user derived from a three-dimensional data set with or without regard to the imaging mode of the system.

Display 237 includes an image of tissue 201. As shown in FIG. 18, display 237 includes two images of tissue 201, a tomographic view and an ILD. Display 237 can include indicia to show a relationship between the content of the ILD and the tomographic view such as, for example, longitudinal marker 219 across the tomographic view and showing the section of tissue 201 that the ILD represents. In some embodiments, longitudinal marker 219 comprises axis 117 and is rotatable around axis 117, for example, by mouse drag operations or keys strokes.

Systems and of the invention are configured to receive input from an operator that comprises a selection of a portion of an image in display 237. An operator may select part of an image in display 237 by any method known in the art including dragging a mouse pointer over a portion of the display, touching a touch-sensitive screen, clicking a button to confirm a proposed selection (for example, as automatically generated by a computer program), keying in positional data, or through interacting with one or more markers presented in display 237.

FIGS. 19-25 depict a shape-sensing mechanism according to certain embodiments. Shape-sensing mechanism are discussed in more detail in U.S. Pub. 2007/0065077 to Childers, et al.

FIG. 19 depicts a fiber optic position and shape sensing device 10 of the present invention that includes an optical fiber as part of a multicore optical fiber 20 with at least two fiber cores 30, 40 spaced apart wherein mode coupling between the fiber cores is minimized. In order to achieve optimal results, mode coupling between the fiber cores should be minimized if not completely eliminated. In some embodiments, a shape-sensing mechanism includes 3 fiber optic cores (as depicted in FIG. 18).

In certain embodiments, the optical fiber elements are made by methods that include designing and modeling the optical parameters (i.e. refractive index profile, core diameters, cladding diameters, etc.) to obtain the desired waveguide performance. The fabrication of multicore optical fiber may include the modification of standard over-cladding and fiber fabrication processes. In some embodiments, multi-chuck over-cladding procedure and the stack-and-draw process are used. In those techniques, the original preforms with the desired dopants and numerical aperture are fabricated via vapor deposition (e.g., Modified Chemical Vapor Deposition (MCVD) process). The preforms are then stretched to the appropriate diameters.

Following the preform stretch, the preforms are sectioned to the appropriate lengths and inserted into a silica tube with the other glass rods to fill the voids in the tube. The variation in the two procedures arises in the method in which the preform rods are inserted into the tube. In the multi-chuck method the bait rods and preforms are positioned in the tube on a glass working lathe. A double chuck is used to align the preforms in the tube. Once positioned, the tube is collapsed on the glass rods to form the preform. The preform is then fiberized in the draw tower by a standard procedure known to those of ordinary skill in the art. In the stack-and-draw process, the preforms and the bait rods are positioned together in the silica tube, with the interstitial space filled with additional glass rods. The glass assembly is then drawn into fiber with the appropriate dimensions.

An array of fiber Bragg gratings 50 is disposed within each fiber core. Such array is defined as a plurality of fiber Bragg gratings disposed along a single fiber core. In certain embodiment, the array includes 100 fiber Bragg gratings. Each fiber Bragg grating is used to measure strain on the multicore optical fiber. Fiber Bragg gratings are fabricated by exposing photosensitive fiber to a pattern of pulsed ultraviolet light from an excimer laser, forming a periodic change in the refractive index of the core. This pattern, or grating, reflects a very narrow frequency band of light that is dependent upon the modulation period formed in the core. In its most basic operation as a sensor, a Bragg grating is either stretched or compressed by an external stimulus. This results in a change in the modulation period of the grating which, in turn, causes a shift in the frequency reflected by the grating. By measuring the shift in frequency, one can determine the magnitude of the external stimulus applied.

Referring back to FIG. 19, the multicore optical fiber 20 is coupled to single core optical fibers 55, 57 through a coupling device 25. Preferably, each single core optical fiber 55, 57 has a broadband reference reflector 60 (e.g., FIG. 20) positioned in an operable relationship to each fiber Bragg grating array wherein an optical path length is established for each reflector/grating relationship. However, it is important to note that the broadband reference reflector is not necessary in order for the invention to work. Alternatively, it is well understood in the art that all optical frequency domain reflectometers include a means, such as a reflector, to establish a reference path and, therefore, a separate reflector such as the broadband reference reflector is not an essential element of the invention. Similarly, some optical frequency domain reflectometers rely on an internal reference path, thus eliminating the need for an external broadband reference reflector altogether. As a preferred embodiment, a frequency domain reflectometer 70 is positioned in an operable relationship to the multicore optical fiber 20 through the single core optical fibers such that the frequency domain reflectometer 70 is capable of receiving signals from the fiber Bragg gratings.

In further embodiments of the invention, the array of fiber Bragg gratings are co-located along the multicore optical fiber. The array preferably comprises at least one hundred (100) fiber Bragg gratings. In an alternative embodiment, a wavelength division multiplexing device is positioned in an operable relationship to the multicore optical fiber and is co-located with the frequency domain reflectometer. This arrangement allows for extension of optical fiber length if needed for a specific application, where a much smaller number (less than about one hundred (100) fiber Bragg gratings) are employed.

FIG. 20 depicts an embodiment in which the fiber optic position and shape sensing device 10 has a computer 90 positioned in an operable relationship to the frequency domain reflectometer 70. It is understood that the optical arrangement shown in FIG. 20 is not limited to those devices employing multicore optical fibers but that it may be used in combination with those devices employing single core optical fibers as well. The computer correlates the signals received from the frequency domain reflectometer 70 to strain measurements. These strain measurements are correlated into local bend measurements. A local bend measurement is defined as the bend between a reference sensor and the next set of sensors in the array. The local bend measurements are integrated into a position or shape. If the optical fiber means has only two cores, then shape determination is limited to two dimensions, if there are three or more cores, three dimensional shape is determined, and in both instances, position is determined.

In essence, the present invention operates on the concept of determining the shape of an object by measuring the shape of the optical fiber. Based on these measurements relative position is also ascertainable. For example, shape sensing is accomplished by creating a linear array of high spatial resolution fiber optic bend sensors. Assuming each element is sufficiently small, by knowing the curvature of the structure at each individual element the overall shape is reconstructed through an integration process. A bend sensor is created by adhering two strain sensors to either side of a flexible object or by embedding the sensors in the object. Examples of various objects include but are not limited to: a position tracking device, such as a robot, and flexible objects such as medical instruments or flexible structures. To monitor the shape of an object that can deform in three dimensions, a measure of the full vector strain is required. Hence, a minimum of three cores is preferred with each core containing an array of fiber Bragg grating strain sensors (preferably of at least one hundred (100) fiber Bragg gratings), preferably each sensor collocated in the axial dimension. To form an array of three dimensional bend sensors, it is assumed that, at a minimum, three optical fiber cores are fixed together such that their centers are non-coplanar. Preferably, the core centers are each 120° with respect to each of the other two core centers and form a triangular shape. It should be acknowledged that any number of optical fiber cores greater than three can also be used for three dimensional bend sensing. The separate cores of the optical fiber containing the fiber Bragg grating strain sensor arrays are embedded into a monolithic structure. By co-locating these strain sensors down the length of the structure whereby sensing points are created, the differential strain between the cores is used to calculate curvature along the length of the structure. By knowing the curvature of the structure at each individual sensing point the overall shape of the structure is reconstructed, presuming that each individual sensing point is sufficiently small.

Strain values for each segment of an object (such as a tether) are used to compute a bend angle and bend radius for each segment of the object. Starting from the beginning of the object, this data is then used to compute the location of the next sensor triplet along the object and to define a new local coordinate system. An algorithm interpolates circular arcs between each sensor triplet on the object. The geometry of the remainder of the object is determined by repeating the process for each sensor triplet along the length of the object. Since the fiber Bragg gratings in each sensing fiber are collocated, a triplet of strain values at evenly spaced segments along the object exists. For each step along the object, a local coordinate system (x′, y′, z′) is defined called the sensor frame.

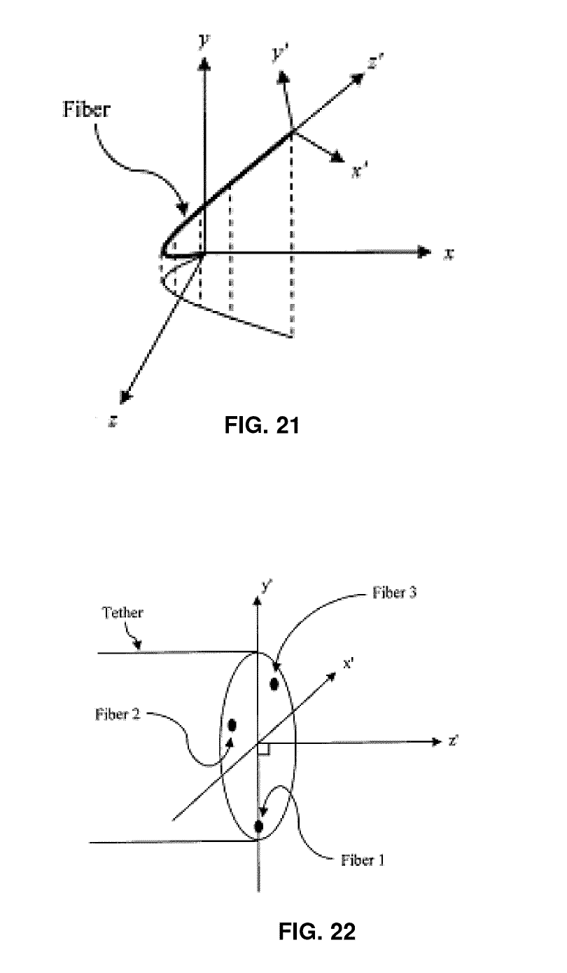

FIG. 21 illustrates the local coordinate system (x′, y′, z′) defined at a specific step along the Fiber object (shown within an arbitrary (x, y, z) coordinate system for illustration).

FIG. 22 shows a relationship between the specific step in the Fiber object and the local coordinate system (x′, y′, z′), wherein the illustrated relationship defines the local coordinate system (x′, y′, z′). The local coordinate system (x′, y′, z′) has its origin at the center of the object's perimeter for any given sensor triplet. The z′ axis points in the direction of the object and the y′ axis is aligned with fiber 1. (See FIG. 22.)

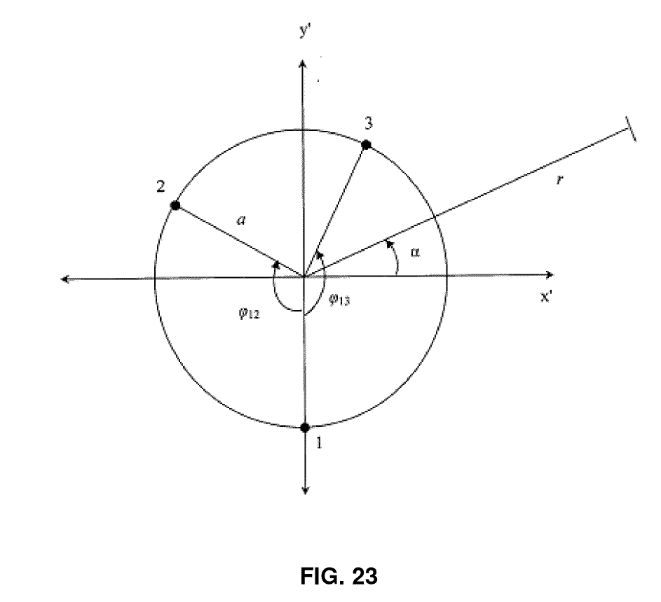

FIG. 23 defines a geometry of a bend, α, and a bend radius, r. Using the three strain values for a given sensor triplet one can calculate the direction of the bend, α, with respect to the x′ axis as well as the bend radius, r, which is the distance from the center of curvature to the center of the core perimeter (see FIG. 22). Knowing r and α for a particular object segment permits the computation of the coordinates of the end of the segment in the (x′, y′, z′) coordinate system. The beginning of the fiber segment is taken to be the origin of the (x′, y′, z′) system. When there is no curvature to the fiber segment, each core segment has a length s.

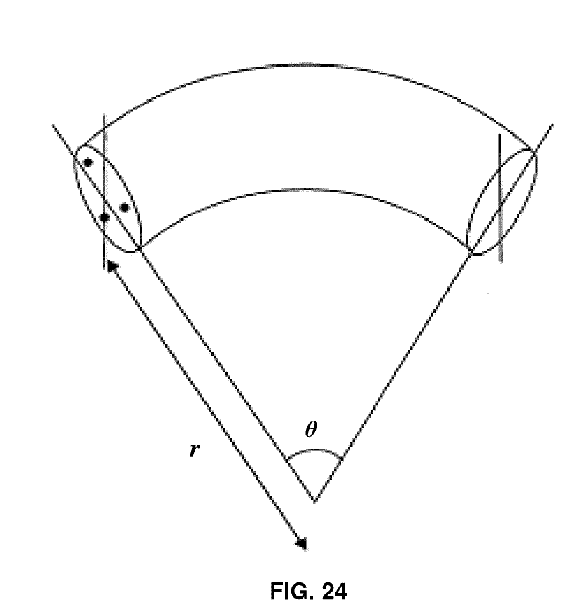

FIG. 24 illustrates curvature of an object and differential curvature of each core therein. When a curvature is introduced, each core is generally a different distance (r1, r2, r3) from the center of curvature, as shown in FIG. 24. Since all of the core segments subtend the same curvature angle, θ, each segment must have a different length.

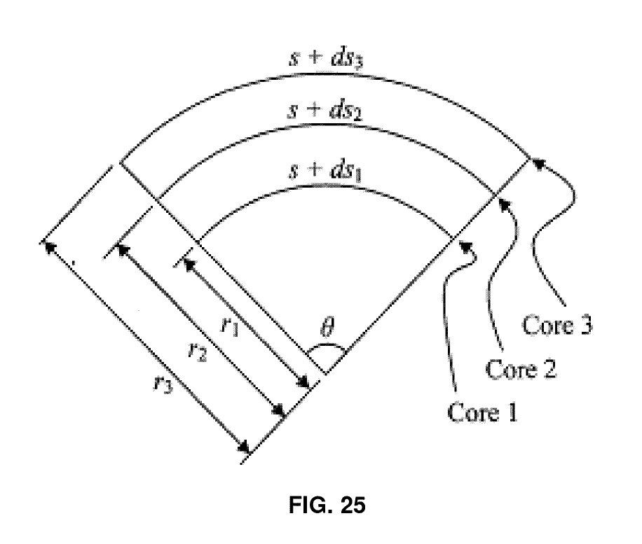

FIG. 25 shows geometrically differing lengths of each core segment subtending the curvature angle θ. The change in length due to bending the fiber is denoted as ds1, ds2 and ds3 as shown in FIG. 23. Additional discussion of shape-sensing may be found in U.S. Pat. No. 8,050,523 to Younge; U.S. Pat. No. 8,047,996 to Goodnow; U.S. Pat. No. 7,720,322 to Prisco; U.S. Pub. 2012/0323075 to Younge; and U.S. Pub. 2006/0013523 to Childlers, the contents of each of which are incorporated by reference. Including shape-sensing information with intravascular imaging information provides high quality 3D (or higher) images.

FIG. 26 is a 2D projection of a vessel border. Here, a shape of an imaging probe that was detected by the probe was not used to create the image. That is, the 3D or 4D shape data is in a file but not being interpreted into the image (an option for users who may so prefer). Since the real 3D shape is not being interpreted into the image, the rendering treats the catheter axis as linear.

FIG. 27 is a 3D representation of a vessel border. Here, real 3D (or higher) sensed shape information is interpreted into the image. The image is 3D in the sense that, as displayed on a computer monitor, a user could pan, scroll, zoom, rotate, or perform other operations, and the depicted vessel would follow the user's input accordingly. The data collected by shape sensing is used to transform the data collected by the intravascular imaging operation. For example, where an OCT system is used, each pixel is transformed in position according to a deviation of the imaging tip from a linear axis.

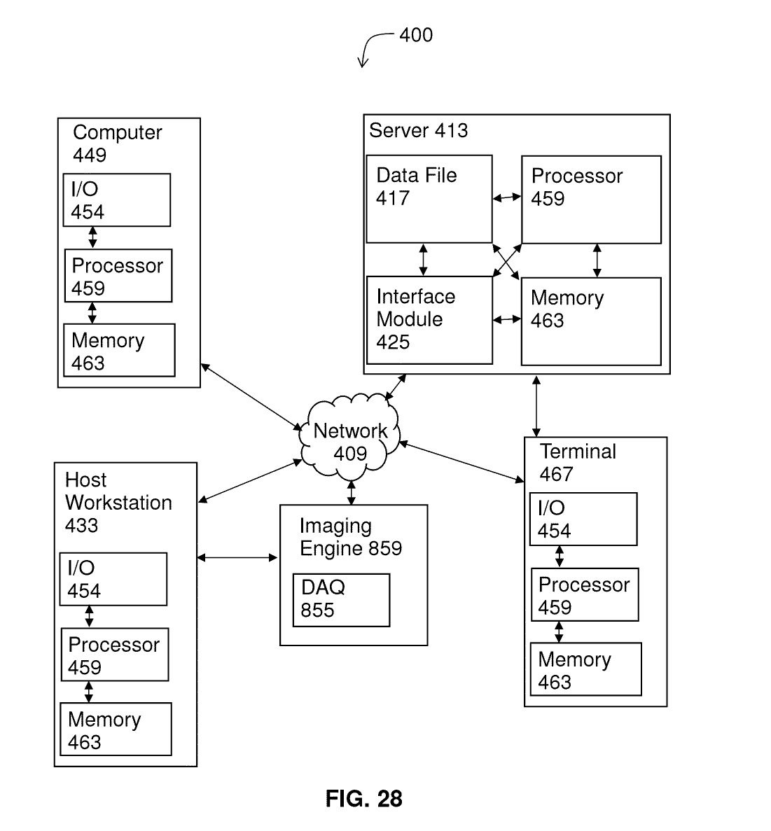

FIG. 28 diagrams a system 400 for using a shape-sensing intravascular interventional catheter according to embodiments disclosed herein. As shown in FIG. 26, imaging engine 859 communicates with host workstation 433 as well as optionally server 413 over network 409. In some embodiments, an operator uses computer 449 or terminal 467 to control system 400 or to receive images. Each of computer 449, server 413, terminal 467, and host workstation 433 may be a computing device according to certain embodiments of the invention. An image may be displayed using an I/O 454, which may include a monitor. Any I/O may include a keyboard, mouse or touchscreen to communicate with any of processor 459, for example, to cause data to be stored in any tangible, nontransitory memory 463. Server 413 generally includes an interface module 425 to effectuate communication over network 409 or write data to data file 417.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for examining tissue comprising:
using an intravascular probe to evaluate bodily material; and
determining a shape of the intravascular probe using a shape-sensing mechanism of the intravascular probe.

2. The method of claim 1, wherein evaluating the bodily material comprises obtaining and storing in a tangible memory coupled to a processor within a computing device a three-dimensional data set representing tissue.

3. The method of claim 1, further wherein the probe is part of an ultrasound image collection system.

4. The method of claim 1, wherein the shape-sensing mechanism comprises at least two fiber cores and an array of fiber Bragg gratings disposed within each fiber core.

5. The method of claim 4, wherein the array of fiber Bragg gratings are substantially collocated along each fiber core.

6. The method of claim 1, wherein the shape-sensing mechanism comprises three non-coplanar optical fibers.

7. The method of claim 1, wherein evaluating the bodily material comprises one selected from the list consisting of: measuring fractional flow reserve; and performing an intra-vascular ultrasound imaging operation; photoacoustic imaging.

8. The method of claim 1, wherein evaluating the bodily material comprises performing an intravascular imaging operation to obtain a three-dimensional data set representing tissue.

9. The method of claim 8, further comprising using the determined shape to present a provide a three-dimensional view of the three-dimensional data set representing tissue.

10. The method of claim 1, wherein the probe comprises an imaging catheter, the method further comprising:
performing, using the catheter, an intravascular imaging operation to obtain a three-dimensional data set representing tissue;
using the determined shape to correct a distortion in the three-dimensional data set.

11. The method of claim 1, wherein the intravascular probe comprises an optical fiber and the shape-sensing mechanism comprises the optical fiber.

12. The method of claim 11, further comprising imaging tissue within a vessel using the optical fiber.

13. The method of claim 12, wherein the shape-sensing mechanism comprises one or more fiber Bragg gratings.

14. A catheter-based sensing apparatus comprising:
an elongated catheter body;
a fiber optic member extending along the body and configured to detect a shape of the body;
an intravascular sensing device; and
an imaging engine comprising a memory coupled to a processor and operable to receive shape information from the fiber optic member and an intravascular image of tissue from the sensing device.

15. A system for examining tissue comprising:
an intravascular probe comprising an imaging mechanism configured for intravascular imaging;
a shape-sensing mechanism configured to determine a shape of the probe using the probe; and
a computing device comprising a non-transitory memory coupled to a processor and operable to receive and store a three-dimensional data set representing tissue captured by the imaging mechanism.

16. The system of claim 15, wherein the shape-sensing mechanism comprises three non-coplanar optical fibers.

17. The system of claim 15, further operable to perform an intravascular imaging operation to obtain a three-dimensional data set representing tissue.

18. The system of claim 17, further operable to use the determined shape to provide a three-dimensional view of the three-dimensional data set representing tissue.

19. The system of claim 15, further operable to:
perform, using the catheter, an intravascular imaging operation to obtain a three-dimensional data set representing tissue;
use the determined shape to correct a distortion in the three-dimensional data set.

20. The system of claim 15, wherein the intravascular probe comprises an optical fiber and the shape-sensing mechanism comprises the optical fiber.
